(19) United States
(12) Patent Application Publication
(10) Pub. No.: US 2007/0128285 A1
(43) Pub. Date: Jun. 7, 2007

(54) Pharmaceutical composition for oral administration

(76) Inventors: Chikara Jin, Saitama-shi (JP); Noboru Tatsumi, Takamatsu-shi (JP); Masatake Dairaku, Saitama-shi (JP); Fuminori Fukushima, Saitama-shi (JP); Toshio Shimizu, Saitama-shi (JP); Mitsuo Togashi, Saitama-shi (JP); Hiroshi Ninomiya, Saitama-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/566,829

(22) PCT Filed: Jul. 12, 2005
(86) PCT No.: PCT/JP05/12835

§ 371 Date: Jan. 31, 2006

(51) Int. Cl. A61K 31/538; A61K 31/4745; A61K 9/14

(52) U.S. Cl. 424/488; 514/230.5; 514/305

(57) ABSTRACT

Provided is a pharmaceutical composition for oral administration containing a 5-HT.₃ receptor antagonist, which is suitable for self-medication because of good preservation stability, low synthesis, good uniformity and good external appearance, and good smoothness in throat, and easiness to be taken. Concretely, it is a jellied pharmaceutical composition for oral administration containing a 5-HT.₃ receptor antagonist, a gelatinizing agent, and water; and having a pH of 7 or less. In particular, there is provided the pharmaceutical composition, where the gelatinizing agent is carrageenan, pectin, agar, alginic acid, sodium alginate, gelatin, manna, Kodak, konjakmannan, glucomannan, chitosan, xanthan gum, tamarind seed polysaccharide, gellan gum, karaya gum, or cassia gum, or preferably the pharmaceutical composition further containing a thickener.

### TECHNICAL FIELD

The present invention relates to a jellied pharmaceutical composition for oral administration containing a 5-HT.₃ receptor antagonist. More specifically, the present invention relates to a jellied pharmaceutical composition for oral administration, which can be easily taken by cancer patients, elderly individuals, or patients with dysphagia, while securing the preservation stabilities of the composition with respect to, for example, exterior appearance and homogeneity thereof.

### BACKGROUND ART

For patients (cancer patients) administered of antineoplastics, side effects such as retching/vomiting, dry mouth accompanied with decreased salivary secretion, stomatitis, glossitis, angular cheilitis, asitia, epigastric distress, stomachache, and so on, are great torments, and particularly vomiting is the severest side effect for cancer patients. In addition, particularly for elderly cancer patients or those with dysphagia among the cancer patients, one of problems is to very hard to take medicines due to dry mouth or decreased salivary secretion.

The 5-HT.₃ receptor antagonists have excellent inhibitory effects on retching and vomiting, which are the side effects of administration of antineoplastics. The 5-HT.₃ receptor antagonists have been mainly used as injections having expected immediate effectivities. There is no problem when the injections are used for hospitalized cancer patients. On the other hand, it is difficult for cancer patients after discharge from hospitals to use the injections for self-medication in home or in an ambulatory setting. Therefore, antiemetic drugs (preferably, preparations of 5-HT.₃ receptor antagonists) against retching and vomiting to be taken by (orally administrated to) a cancer patient after discharge from a hospital, who may take the administration of an antineoplastic, have been required.

In other words, the development of antiemetic drugs (e.g., preparations of 5-HT.₃ receptor antagonists) against retching and vomiting, which are preparations to be taken without anxiety by cancer patients after discharge from hospitals, particularly elderly individuals or patients with dysphagia, have been expected.

In most clinical cases, those antiemetic drugs are orally administered one or two hours before the administration of antineoplastics, routinely.

Generally, oral preparations such as tablets and fine grain agents have been applied for self-medication. For elderly or dysphagia individuals who have decreased swallowing abilities, those dosage forms are those which can be hardly taken. Besides, those dosage forms are those which can be very difficult to be taken against hypersensitive vomiting action due to administration of antineoplastics. Therefore, tables which can be quickly decayed or dissolved with saliva in the mouth have been developed as solid preparations which can be easily taken by those patients. However, many of cancer patients, elderly, or dysphagia individuals are accompanied with dry mouth by a decrease in salivary secretion function. For such patients, therefore, it is hard to say that the tablet which can be quickly decayed or dissolved with saliva in the mouth may have a dosage form with improved dose characteristics.

Meanwhile, a jellied pharmaceutical composition for oral administration has been also known as a dosage form having improved dose characteristics. For instance, a jellied pharmaceutical composition for oral administration, characterized in excellent stability and good smoothness in throat as well as being difficult to undergo synthesis (JP-A-9-187233) and a container that allows a jellied pharmaceutical composition for oral administration to be easily administered at once (JP-A-9-194346) have been reported.

### DISCLOSURE OF THE INVENTION

The present invention intends to provide a preparation of a jellied pharmaceutical composition for oral administration, containing a 5-HT.₃ receptor antagonist as a main drug and having ensured preservation stabilities, good flavor, and good smoothness in throat, which can be administered at once with simple handling.

The present invention thus intends to provide a preparation that can be easily taken by a cancer patient (particularly, a patient with dry mouth caused by decreased salivary secretion function) and can prevent side effects (retching/vomiting) due to an antineoplastic drug.

As a result of intensive studies in light of the circumstances described above, the inventors of the present invention found that a jellied preparation containing a 5-HT.₃ receptor antagonist, a gelatinizing agent, and water, and having a pH 7 or less has excellent preservation stabilities, good flavor, and good smoothness in throat and can be easily taken by a cancer patient (particularly, a patient with dry mouth caused by decreased salivary secretion function) and can prevent side effects (retching/vomiting) due to an antineoplastic drug.

That is, the present invention is as follows.
[1] A jellied pharmaceutical composition for oral administration, containing a 5-HT.₃ receptor antagonist, a gelatinizing agent, and water, and having a pH of 7 or less.
[2] The pharmaceutical composition for oral administration according to the above item [1], further containing a reductant.
[3] The pharmaceutical composition for oral administration according to the above item [1] or [2], in which the 5-HT.₃ receptor antagonist is azasetron, granisetron, tropisetron, ramosetron or ondansetron, or an organic acid salt or inorganic acid salt thereof.
[4] The pharmaceutical composition for oral administration according to the above item [1] or [2], in which the gelatinizing agent is carrageenan, pectin, agar, alginic acid, sodium alginate, gelatin, manna, Kodak, konjakmannan, glucomannan, chitosan, xanthan gum, tamarind seed polysaccharide, gellan gum, karaya gum or cassia gum, or a combination of two or more of them.
[5] The pharmaceutical composition for oral administration according to the above item [1] or [2], in which the gelatinizing agent includes carrageenan and the carrageenan is kappa (κ)-carrageenan and/or iota (ι)-carrageenan.
[6] The pharmaceutical composition for oral administration according to the above item [1] or [2], further containing a thickener, in which the thickener is locust bean gum, gum arabic, tragacanth, dextrin, dextran, arabinogalactan, pullulan, carmellose sodium, hydropropyl cellulose, hydroxyethyl methyl cellulose, methyl cellulose, carboxymetyl cellulose, copolydone, polyvinylpyrrolidone, carboxyvinyl polymer, sodium polyacrylate, or macrogol, or a combination of two or more thereof.
[7] The pharmaceutical composition for oral administration according to the above item [1] or [2], further containing a water-soluble salt of potassium or calcium.
[8] A medicine for oral administration, including the pharmaceutical composition for oral administration according to any one of the above items [1] to [7] and a light-blocking type container containing the pharmaceutical composition.

### BEST MODE FOR CARRYING OUT THE INVENTION

The jellied pharmaceutical composition for oral administration of the present invention (hereinafter, also referred to as “the composition of the present invention”) is characterized by containing a 5-H₃ receptor antagonist, a galatinizing agent, and water, and having a pH of 7 or less.

Examples of the 5-HT.₃ receptor antagonist in the composition of the present invention include azasetron, granisetron, tropisetron, ramosetron, and ondansetron (hereinafter, may be generally referred to as “setron drugs”). In the composition, the 5-HT.₃ receptor agonist (preferably, a setron drug) is present in the form of a salt with a cation (e.g., hydrochloride), a salt with an anion, a free product, or a mixture thereof under the conditions for formulating a preparation (e.g., pH and kind of anion present therein).

The 5-HT.₃ receptor antagonist is generally administered at a dose of about 1 to 11 mg per once (depending on the type of the 5-HT.₃ receptor antagonist). On the other hand, as a rough guide, the jelly agent of the present invention is about 0.5 to about 10 g in weight, which can be easily administered (e.g., administered in one mouthful). Therefore, the content of the 5HT.₃ receptor antagonist is preferably in the range of 0.001 to 20% by mass, more preferably in the range of 0.01 to 10% by mass with respect to the total amount of the composition.

Examples of the gelatinizing agent in the pharmaceutical composition for oral administration of the present invention include carrageenan, pectin, agar, alginic acid, sodium alginate, gelatin, manna, Kodak, konjakmannan, glucomannan, chitosan, xanthan gum, tamarind seed polysaccharide, gellan gum, karaya gum, cassia gum, tara gum, guar gum, psyllium seed gum, and ghatti gum, and used alone or a combination of two or more of them.

The amounts of those gelatinizing agents added with respect to the total amounts of the jellied composition are 0.01 to 7% by mass, more preferably 0.05 to 5% by mass, and still more preferably 0.1 to 3% by mass.

At least one part of the gelatinizing agent contained in the composition of the present invention is preferably carrageenan or pectin (particularly carrageenan), because a combination of carrageenan or pectin with a thickener (particularly, locust bean gum) can improve the characteristics of the composition, as described later.

Carrageen an is a polysaccharide extracted from marine algae and can be grouped into three types: kappa (κ), iota (ι), and lamda (λ) depending on the difference of the amounts of sulfate groups and anhydro groups in molecule. Carrageen an in the composition of the present invention may be any of types, preferably kappa-type carrageenan or iota-type carrageenan, or a mixture thereof.

The present inventors have found that 5-HT.₃ receptor antagonists (preferably, setron drugs), organic acid salts or inorganic acid salts thereof do not inhibit the formation of-a jellied gel of kappa- or iota-type carrageenan. Carrageen an is dissolved in water as a random coil molecule by dispersing the carrageenan in water, and heating at about 60° C. or more. When this solution is cooled, double helices of the carrageenan can be formed by intermolecular association and it is then provided as a junction zone to form a jellied gel. However, the formation of the jellied gel may not be occurred, as the 5-HT.₃ receptor antagonists (preferably setron drugs), organic acid salts or inorganic acid salts thereof may inhibit the formation of double helices of lamda-type carrageenan.

Therefore, as described above, the carrageenan in the composition of the present invention is preferably kappa-type carrageenan or iota-type carrageenan, or a mixture thereof.

The content of carrageenan (preferably, kappa- and/or iota-type(s)) in the composition of the present invention is 0.02 to 5.0% by mass, more preferably 0.03 to 3.0% by mass, still more preferably 0.05 to 1.5% by mass in total with respect to the total amount of the composition.

As described above, the composition of the present invention is characterized by having a pH of 7 or less. The pH is preferably in the range of 3 to 7, more preferably in the range of 5 to 7. This is because, in the composition having the pH in such a range, a 5-HT.₃ receptor antagonist (preferably, a setron drug) can be present in stable.

The present inventors have investigated the stability of each setron drug (azasetron, granisetron, tropisetron, ramosetron, or ondansetron) in the aqueous solution of pH 8 to3. That is, an aqueous solution of each setron drug (0.1 w/w %) was adjusted to various pH values (pH 8 to 3) with hydrochloric acid or sodium hydroxide. After storing at 40° C. for three months, the external appearance and taste of the solution were observed. As a result, for each of the setron drugs, the aqueous solution of pH 8 is colored faint yellow, but the aqueous solution of pH 3 to 7 is colorless. It is found that the setron drug can be stable in the aqueous solution of pH 3to 7. As similar to this result, the 5-HT.₃ receptor antagonist (preferably the setron drug) is also present stably in an aqueous gel of pH 3 to 7 (preferably pH 5 to 7).

Therefore, the composition of the present invention is stably adjusted to the above pH range, so that it may contain a pH regulator and/or a buffer. Concrete examples of the pH regulator include organic acid salts such as citric acid and salts thereof, phosphoric acid and salts thereof, dilute hydrochloric acid, tartaric acid, dl-malic acid, and succinic acid. Concrete examples of the buffer include acids such as citric acid, glutamic acid, tartaric acid, dl-malic acid and succinic acid, and metallic salts thereof.

The composition of the present invention can be almost satisfied with its quality for a jellied pharmaceutical composition for oral administration as far as it may contain a 5-HT.₃ receptor antagonist or a salt thereof (an organic acid salt or inorganic acid salt), a gelatinizing agent (preferably, kappa- or iota-type carrageenan, or pectin), and water. However, for further enhancing formability, lowering synthesis property, or attaining more favorable quality, the composition of the present invention may contain any ingredient. Examples of any ingredient include thickeners, aqueous salts of potassium or sodium, reductants, polyalcohols, buffers, antiseptic agents, sweeteners, and flavors.

The composition of the present invention preferably contains a thickener out of the above optional ingredients. Examples of the thickener include locust bean gum, gum arabic, tragacanth, dextrin, dextran, arabinogalactan, pullulan, carmellose sodium, hydropropyl cellulose, hydroxyethyl methyl cellulose, methyl cellulose, carboxymetyl cellulose, copolydone, polyvinylpyrrolidone, carboxyvinyl polymer, sodium polyacrylate, or macrogol, and may be used alone or a combination of two or more thereof.

Of the above thickeners, locust bean gum of galactomannan polysaccharide extracted from Carob tree is preferably exemplified. Because a combination of the locust bean gum and the carrageenan of a gelatinizing agent can provide a composition having higher jelly strength and lower synthesis property. This could be because a strong gel can be formed by the formation of a complex junk zone as a result of association of the double helix portion of carrageenan with the manna portion of locust bean gum of the galactomannan polysaccharide as thickener.

In addition, a combination of the locust bean gum and pectin can provide a jellied composition having lower synthesis property.

The content of the thickener in the composition of the present invention may be suitably selected depending on the type of the thickener. However, as a rough guide, it may be in the range of about 0.02 to 5% by mass, more preferably in the range of about 0.03 to 3% by mass, still more preferably in the range of about 0.05 to 2% by mass with respect to the total of the composition. For instance, in the case of locust bean gum, it is in the range of 0.05 to 2% by mass.

A further reinforcement in gel is attained by containing a thickener, so that a jellied composition with improving fragile and synthesis proclivities can be obtained.

The composition of the present invention preferably also includes a divalent metal ion such as a calcium ion, a trivalent metalic ion, or a potassium ion. The gelatinization of the composition of the present invention is promoted by the above metal ion, when in particular at least part of a gelatinizing agent incorporated in the composition is pectin, alginic acid, sodium alginate, manna, glucomannnan, carageenan, xanthan gum, tamarind seed polysaccharide, gellan gum, karaya gum, cassia gum, tara gum, guar gum, psyllium seed gum, ghatti gum, or the like.

Of the above metal ions, it is found that the inclusion of water-soluble salts of the metal ion such as a calcium or potassium ion (inorganic acid salt such as chloride, phosphate or sulfuric acid, or organic acid salt such as lactic acid or citric acid) into the composition is effective to jellify the composition and enhance the jelly-stability.

In addition, the salt such as calcium or potassium can impart any strength to the jellied preparation of the present invention. Therefore, the use of those salts may produce jellied preparation having different texture (such as chewiness) depending upon patient's tooth.

The content of the metal ion salt in the composition of the present invention varies depending on the type and quantity of the gelatinizing agent in the composition. For example, when the gelatinizing agent is carrageenan, the content is in the range of 1 to 15% by mass, more preferably 2to 12% by mass, still more preferably 4 to 10% by mass with respect to the amount of carrageenan added.

The composition of the present invention preferably contains a reductant out of the above optional ingredients. Examples of the reductant include sodium pyrosulfite, sodium sulfite, vitamin E, BHA, BHT, ascorbic acid, cysteine hydrochloride, sodium thioglycolate, sodium thiomalate, and sodium thiosulfate. Preferably, the reductant includes sodium pyrosulfite or ascorbic acid in view of general versatility and cost.

The inclusion of the reductant can reduce the influence of oxygen or light on variation with time of property (such as discoloration or the like) of the pharmaceutical preparation for oral administration, the composition of the present invention. The content of the reductant in the composition can be suitably determined depending on the type thereof, and defined on the basis of the range of the amount allowable for a pharmaceutical additive for internal. preparations.

Furthermore, as represented in test examples described below, the composition of the present invention produced in the presence of the reductant may be prevented from discoloration. It is considered that the. discoloration may be due to decomposition of the 5-HT.₃ receptor antagonist. For preventing the discoloration, the presence of the reductant in the heating process of the production of the composition is particularly preferable.

As described above, the jellied composition of the present invention may contain a polyalcohol, a sweetener, a flavor, an antiseptic agent, and so on for conditioning the qualities thereof, such as taste, flavor, smoothness, and ease of swallowing.

Examples of the polyalcohol include glycerin, propylene glycol, D-sorbitol, xylitol, mannitol, erythritol, and sucralose.

Examples of the sweetener include fructose, purified sucrose, palatinose, trehalose, oligosaccharide, aspartame, isomerized sugar, fructose, muscovado, saccharin, saccharin sodium, sweet hydrangea leaf, powdered sweet hydrangea leaf, stevioside, licorice, licorice extract, glucose, starch syrup, powdered starch syrup, reduced maltose starch syrup, and powdered sticky rice.

Examples of the flavor include fennel, fennel oil, orange, orange extract, orange essence, orange oil, mentha water, mentha oil, honey, d-borneol, dl-menthol, l-menthol, eucalyptus oil, lavender oil, lemon oil, rose oil, sugar flavor, vanilla flavor, vanillin, chocolate flavor A22736, fruit flavor, cherry flavor, ethyl vanillin, and various fruit juices.

Example of the antiseptic includes one recognized as a pharmaceutical additive such as sodium benzoate, sodium edetate, sodium salicylate, sorbic acid, sodium dehydroacetate, isobutyl parahydroxybenzoate, isopropyl parahydroxybenzoate, ethyl parahydroxybenzoate, butyl parahydroxybenzoate, propyl parahydroxybenzoate, or methyl parahydroxybenzoate.

The amount of each of those ingredients to be added is determined on the basis of the range of an amount permitted in their actual practical uses as pharmaceutical additives in internal preparations.

The composition of the present invention is preferably a medicine preparation for oral administration by filling in a disposable container suitable for self-medication. The disposable container and the filling method can be used as those described in JP-A-9-194346.

In addition, the container is preferably of a light-blocking type. Example of the light-blocking type containers include colored containers such as bister.

The composition of the present invention may be produced in accordance with a conventional method of producing a jellied composition, except that compounding 5-HT.₃ receptor antagonist into the composition. The medicine for oral administration of the present invention can be produced by incorporating the composition into the container.

For instance, the composition of the present invention can be produced by stirring a mixture of a 5-HT.₃ receptor antagonist, a gelatinizing agent, and water. Preferably, it may be produced by stirring a mixture of a 5-HT.₃ receptor antagonist, a gelatinizing agent, a reductant, and water.

Concretely, for example, the composition of the present invention may be produced according to the following steps, but not limited to the steps.

First step: Purified water and a buffer are check-weighed and placed into a preparation tank and dissolved with stirring at room temperature or on heating.

Second step: A pH adjuster is added.

Third step: A 5-HT.₃ receptor antagonist and a reductant are added and then heated to dissolve with stirring.

Fourth step: A gelatinizing agent and a thickener as required are added and then heated to dissolve with stirring.

Fifth step: An antiseptic agent, a flavor, a sweetener, and so on are added and then sterilized by heating for one hour.

Sixth step: The drug solution in the preparation tank is dispensed and filled into disposal containers for a single administration suitable for self-medication, with keeping the temperature of the drug solution in the preparation tank on heating.

Seventh step: The drug solution in the container is cooled and solidified with a cooling apparatus, and then the container is subjected to pillow-packing with a packing machine, and thereby to be a medicine for oral administration.

A kind and a blending quantity of the ingredients (such as the 5-HT.₃ receptor antagonist, the gelatinizing agent, and the reductant) to be used in each of the steps may be the same as those of the ingredients of the above composition of the present invention.

The 5-HT.₃ receptor antagonist to be used in the third step is preferably a setron drug (such as azasetron, granisetron, tropisetron, ramosetron, or ondansetron), and typically added as a hydrochloride.

The heating temperatures in the steps 3 to 5 are preferably in the range of 60 to 95° C. more preferably in the range of 80 to 90° C.

The addition of a reductant is preferable to carry out simultaneously with the addition of the 5HT.₃ receptor antagonist (the third step) or immediately after that. In other words, the heating step in the production of the composition of the present invention is preferably carried out in the presence of the reductant.

Hereinafter, with reference to examples and test examples, the present invention will be further described in detail. However, the scope of the present invention is not limited to those examples.

### EXAMPLES

### Example 1

A pharmaceutical preparation for oral administration of jellied composition was produced according to the above-mentioned preparation process.

That is, in a preparation tank, a mixture of purified water (107.23 g), citric acid (0.12 g), and sodium citrate (1 g) was dissolved by stirring at room temperature or at ambient temperature. The solution was at pH 6.5. Then, granisetron hydrochloride (147.4 mg) and sodium pyrosulfite (0.1 g) were added to the solution and then dissolved by stirring at 80 to 90° C. In addition, kappa-carrageenan (0.4 g), iota-carrageenan (0.9 g), locust bean gum (0.4 g), dextrin (5 g), and sodium polyacrylate (4 mg) were added to the solution and then dissolved by stirring at 80 to 90° C. Furthermore, the solution was added with D-sorbitol (56 g), glycerin (27 g), and propyl parahydroxybenzoate (0.5 g), and flavor (trace amount), and then added properly with purified water was so as to be adjusted to 198 g in total, followed by subjecting to sterilization treatment with heating at 80 to 90° C. for one hour.

While keeping the temperature of the drug solution in the preparation tank, the solution (3 g) was dispensed and filled into disposal container for a single administration. After dispensation, it was cooled and solidified with a cooling apparatus, and then subjected to pillow packing with a packing machine.

### Examples 2 to 34 and Comparative Example 1

Hereinafter, just as in the case with Example 1, pharmaceutical preparations for oral administration of jellied compositions of Examples2to 34 and Comparative Example 1 were respectively prepared, according to the formulas shown in table 1 to 6.

TABLE 1

| | Blending quantity | | | | | | | | | |
|---|---|---|---|---|---|---|---|---|---|---|
| Ingredients and pH | Example 1 | | Example 2 | | Example 3 | | Example 4 | | Example 5 | |
| 5-HT₃ receptor antagonist | granisetron | | azasetron | | Tropisetron | | ramosetron | | ondansetron | |
| | hydrochloride | | hydrochloride | | hydrochloride | | hydrochloride | | hydrochloride | |
| | 147.4 mg | | 728.8 mg | | 372.2 mg | | 66 mg | | 330 mg | |
| kappa-carrageenan | 0.4 | g | 0.4 | g | 0.4 | g | 0.4 | g | 0.4 | g |
| (manufactured by Sansho Co., Ltd.) | | | | | | | | | | |
| iota-carrageenan | 0.9 | g | 0.9 | g | 0.9 | g | 0.9 | g | 0.9 | g |
| (manufactured by Sansho Co., Ltd.) | | | | | | | | | | |
| locust bean gum | 0.4 | g | 0.4 | g | 0.4 | g | 0.4 | g | 0.4 | g |
| (CP Kelco Co., Ltd., San-Ei Gen | | | | | | | | | | |
| F.F.I., Inc.) | | | | | | | | | | |
| Dextrin | 5 | g | 5 | g | 5 | g | 5 | g | 5 | g |
| (Japanese Pharmacopoeia 14: Nippon | | | | | | | | | | |
| Starch Chemical Co., Ltd.) | | | | | | | | | | |
| citric acid | 0.12 | g | 0.12 | g | 0.12 | g | 0.12 | g | 0.12 | g |
| (Japanese Pharmacopoeia 14) | | | | | | | | | | |
| sodium citrate | 1 | g | 1 | g | 1 | g | 1 | g | 1 | g |
| (Japanese Pharmacopoeia 14) | | | | | | | | | | |
| sodium polyacrylate | 4 | mg | 4 | mg | 4 | mg | 4 | mg | 4 | mg |
| (manufactured by Nihon Junyaku | | | | | | | | | | |
| Co., Ltd.) | | | | | | | | | | |
| D-sorbitol | 56 | g | 56 | g | 56 | g | 56 | g | 56 | g |
| (Japanese Pharmacopoeia 14) | | | | | | | | | | |
| glycerin | 27 | g | 27 | g | 27 | g | 27 | g | 27 | g |
| (Japanese Pharmacopoeia 14) | | | | | | | | | | |
| sodium pyrosulfite | 0.1 | g | 0.1 | g | 0.1 | g | 0.1 | g | 0.1 | g |
| (manufactured by Daito Chemical | | | | | | | | | | |
| Co., Ltd.) | | | | | | | | | | |
| propyl parahydroxybenzoate | 0.5 | g | 0.5 | g | 0.5 | g | 0.5 | g | 0.5 | g |
| (Japanese Pharmacopoeia 14) | | | | | | | | | | |

| | | | | | |
|---|---|---|---|---|---|
| Flavor | trace amount | trace amount | trace amount | trace amount | trace amount |
| pH | 6.5 | 6.5 | 6.5 | 6.5 | 6.5 |

Purified water was added to prepare 198 g in total and then filled in a container.

TABLE 2

| | Blending quantity | | | | | | | | | |
|---|---|---|---|---|---|---|---|---|---|---|
| Ingredients and pH | Example 6 | | Example 7 | | Example 8 | | Example 9 | | Example 10 | |
| 5-HT₃ receptor antagonist | granisetron | | azasetron | | tropisetron | | ramosetron | | ondansetron | |
| | hydrochloride | | hydrochloride | | hydrochloride | | hydrochloride | | hydrochloride | |
| | 147.4 mg | | 728.8 mg | | 372.2 mg | | 66 mg | | 330 mg | |
| kappa-carrageenan | 0.4 | g | 0.4 | g | 0.4 | g | 0.4 | g | 0.4 | g |
| (manufactured by Sansho Co., Ltd.) | | | | | | | | | | |
| iota-carrageenan | 0.9 | g | 0.9 | g | 0.9 | g | 0.9 | g | 0.9 | g |
| (manufactured by Sansho Co., Ltd.) | | | | | | | | | | |
| locust bean gum | 0.4 | g | 0.4 | g | 0.4 | g | 0.4 | g | 0.4 | g |
| (CP Kelco Co., Ltd., San-Ei Gen | | | | | | | | | | |
| F.F.I., Inc.) | | | | | | | | | | |
| hydroxypropylcellulose | 2 | g | 2 | g | 2 | g | 2 | g | 2 | g |
| (manufactured by Shin-Etsu Chemical | | | | | | | | | | |
| Co., Ltd.) | | | | | | | | | | |
| citric acid | 0.2 | g | 0.2 | g | 0.2 | g | 0.2 | g | 0.2 | g |
| (Japanese Pharmacopoeia 14) | | | | | | | | | | |
| disodium hydrogen phosphate | 2 | g | 2 | g | 2 | g | 2 | g | 2 | g |
| saccharin sodium (Japanese | 0.132 | mg | 0.132 | g | 0.132 | g | 0.132 | g | 0.132 | g |
| Pharmacopoeia 14) | | | | | | | | | | |
| D-sorbitol | 56 | g | 56 | g | 56 | g | 56 | g | 56 | g |
| (Japanese Pharmacopoeia 14) | | | | | | | | | | |
| glycerin | 27 | g | 27 | g | 27 | g | 27 | g | 27 | g |
| (Japanese Pharmacopoeia 14) | | | | | | | | | | |
| ascorbic acid (Japanese | 0.1 | g | 0.1 | g | 0.1 | g | 0.1 | g | 0.1 | g |
| Pharmacopoeia 14) | | | | | | | | | | |
| propyl parahydroxybenzoate | 0.5 | g | 0.5 | g | 0.5 | g | 0.5 | g | 0.5 | g |
| (Japanese Pharmacopoeia 14) | | | | | | | | | | |

| flavor | trace amount | trace amount | trace amount | trace amount | trace amount |
|---|---|---|---|---|---|
| pH | 6 | 6 | 6 | 6 | 6 |

Purified water was added to prepare 198 g in total and then filled in a container.

TABLE 3

| | Blending quantity | | | | | | | | | |
|---|---|---|---|---|---|---|---|---|---|---|
| Ingredients and pH | Example 11 | | Example 12 | | Example 13 | | Example 14 | | Example 15 | |
| 5-HT₃ receptor antagonist | granisetron | | azasetron | | tropisetron | | ramosetron | | ondansetron | |
| | hydrochloride | | hydrochloride | | hydrochloride | | hydrochloride | | hydrochloride | |
| | 147.4 mg | | 728.8 mg | | 372.2 mg | | 66 mg | | 330 mg | |
| kappa-carrageenan | 0.4 | g | 0.4 | g | 0.4 | g | 0.4 | g | 0.4 | g |
| (manufactured by Sansho Co., Ltd.) | | | | | | | | | | |
| iota-carrageenan | 0.9 | g | 0.9 | g | 0.9 | g | 0.9 | g | 0.9 | g |
| (manufactured by Sansho Co., Ltd.) | | | | | | | | | | |
| gua gum | 0.4 | g | 0.4 | g | 0.4 | g | 0.4 | g | 0.4 | g |
| (San-Ei Gen F.F.I., Inc.) | | | | | | | | | | |
| casein | 3 | g | 3 | g | 3 | g | 3 | g | 3 | g |
| (manufactured by Sansho Co., Ltd.) | | | | | | | | | | |
| citric acid | 0.12 | g | 0.12 | g | 0.12 | g | 0.12 | g | 0.12 | g |
| (Japanese Pharmacopoeia 14) | | | | | | | | | | |
| sodium citrate | 1 | g | 1 | g | 1 | g | 1 | g | 1 | g |
| (Japanese Pharmacopoeia 14) | | | | | | | | | | |
| pullulan | 10 | g | 10 | g | 10 | g | 10 | g | 10 | g |
| reduced maltose starch syrup | 40 | g | 40 | g | 40 | g | 40 | g | 40 | g |
| (San-Ei Gen F.F.I., Inc.) | | | | | | | | | | |
| glycerin | 27 | g | 27 | g | 27 | g | 27 | g | 27 | g |
| (Japanese Pharmacopoeia 14) | | | | | | | | | | |
| sodium pyrosulfite | 0.1 | g | 0.1 | g | 0.1 | g | 0.1 | g | 0.1 | g |
| (manufactured by Daito Chemical Co., | | | | | | | | | | |
| Ltd.) | | | | | | | | | | |
| propyl parahydroxybenzoate (Japanese | 0.5 | g | 0.5 | g | 0.5 | g | 0.5 | g | 0.5 | g |
| Pharmacopoeia 14) | | | | | | | | | | |

| | | | | | |
|---|---|---|---|---|---|
| flavor | trace amount | trace amount | trace amount | trace amount | trace amount |
| pH | 6 | 6 | 6 | 6 | 6 |

Purified water was added to prepare 198 g in total and then filled in a container.

TABLE 4

| | Blending quantity | | | | | | | | | |
|---|---|---|---|---|---|---|---|---|---|---|
| Ingredients and pH | Example 16 | | Example 17 | | Example 18 | | Example 19 | | Example 20 | |
| 5-HT₃ receptor antagonist | granisetron | | azasetron | | tropisetron | | ramosetron | | ondansetron | |
| | hydrochloride | | hydrochloride | | hydrochloride | | hydrochloride | | hydrochloride | |
| | 147.4 mg | | 728.8 mg | | 372.2 mg | | 66 mg | | 330 mg | |
| pectin | 0.4 | g | 0.4 | g | 0.4 | g | 0.4 | g | 0.4 | g |
| (manufactured by Sansho Co., Ltd.) | | | | | | | | | | |
| iota-carrageenan | 0.9 | g | 0.9 | g | 0.9 | g | 0.9 | g | 0.9 | g |
| (manufactured by Sansho Co., Ltd.) | | | | | | | | | | |
| kappa-carrageenan | 0.4 | g | 0.4 | g | 0.4 | g | 0.4 | g | 0.4 | g |
| (manufactured by Sansho Co., Ltd.) | | | | | | | | | | |
| xanthan gum | 0.2 | g | 0.2 | g | 0.2 | g | 0.2 | g | 0.2 | g |
| (manufactured by Sansho Co., Ltd.) | | | | | | | | | | |
| carmellose sodium | 0.2 | g | 0.2 | g | 0.2 | g | 0.2 | g | 0.2 | g |
| citric acid | 0.3 | g | 0.3 | g | 0.3 | g | 0.3 | g | 0.3 | g |
| (Japanese Pharmacopoeia 14) | | | | | | | | | | |
| disodium hydrogen phosphate | 2 | g | 2 | g | 2 | g | 2 | g | 2 | g |
| (Japanese Pharmacopoeia 14) | | | | | | | | | | |
| D-sorbitol | 40 | g | 40 | g | 40 | g | 40 | g | 40 | g |
| (Japanese Pharmacopoeia 14) | | | | | | | | | | |
| glycerin | 27 | g | 27 | g | 27 | g | 27 | g | 27 | g |
| (Japanese Pharmacopoeia 14) | | | | | | | | | | |
| sodium pyrosulfite | 0.1 | g | 0.1 | g | 0.1 | g | 0.1 | g | 0.1 | g |
| (manufactured by Daito Chemical | | | | | | | | | | |
| Co., Ltd.) | | | | | | | | | | |
| propyl parahydroxybenzoate | 0.5 | g | 0.5 | g | 0.5 | g | 0.5 | g | 0.5 | g |
| (Japanese Pharmacopoeia 14) | | | | | | | | | | |

| | | | | | |
|---|---|---|---|---|---|
| flavor | trace amount | trace amount | trace amount | trace amount | trace amount |
| pH | 5.5 | 5.5 | 5.5 | 5.5 | 5.5 |

Purified water was added to prepare 198 g in total and then filled in a container.

TABLE 5

| | Blending quantity | | | | | | | | | |
|---|---|---|---|---|---|---|---|---|---|---|
| Ingredients and pH | Example 21 | | Example 22 | | Example 23 | | Example 24 | | Example 25 | |
| 5-HT₃ receptor antagonist | granisetron | | azasetron | | tropisetron | | ramosetron | | ondansetron | |
| | hydrochloride | | hydrochloride | | hydrochloride | | hydrochloride | | hydrochloride | |
| | 147.4 mg | | 728.8 mg | | 372.2 mg | | 66 mg | | 330 mg | |
| pectin | 0.4 | g | 0.4 | g | 0.4 | g | 0.4 | g | 0.4 | g |
| (manufactured by Sansho Co., Ltd.) | | | | | | | | | | |
| Agar | 0.4 | g | 0.4 | g | 0.4 | g | 0.4 | g | 0.4 | g |
| iota-carrageenan | 0.9 | g | 0.9 | g | 0.9 | g | 0.9 | g | 0.9 | g |
| (manufactured by Sansho Co., Ltd.) | | | | | | | | | | |
| dextrin | 5 | g | 5 | g | 5 | g | 5 | g | 5 | g |
| (Japanese Pharmacopoeia 14: Nippon | | | | | | | | | | |
| Starch Chemical Co., Ltd.) | | | | | | | | | | |
| citric acid | 0.2 | g | 0.2 | g | 0.2 | g | 0.2 | g | 0.2 | g |
| (Japanese Pharmacopoeia 14) | | | | | | | | | | |
| sodium citrate | 1 | g | 1 | g | 1 | g | 1 | g | 1 | g |
| (Japanese Pharmacopoeia 14) | | | | | | | | | | |
| purified sucrose | 40 | g | 40 | g | 40 | g | 40 | g | 40 | g |
| (Japanese Pharmacopoeia 14) | | | | | | | | | | |
| glycerin | 20 | g | 20 | g | 20 | g | 20 | g | 20 | g |
| (Japanese Pharmacopoeia 14) | | | | | | | | | | |
| sodium pyrosulfite | 0.1 | g | 0.1 | g | 0.1 | g | 0.1 | g | 0.1 | g |
| (manufactured by Daito Chemical | | | | | | | | | | |
| Co., Ltd.) | | | | | | | | | | |
| propyl parahydroxybenzoate | 0.1 | g | 0.1 | g | 0.1 | g | 0.1 | g | 0.1 | g |
| (Japanese Pharmacopoeia 14) | | | | | | | | | | |

| | | | | | |
|---|---|---|---|---|---|
| flavor | trace amount | trace amount | trace amount | trace amount | trace amount |
| PH | 5 | 5 | 5 | 5 | 5 |

Purified water was added to prepare 198 g in total and then filled in a container.

TABLE 6

| | Blending quantity | | | | | | | | | |
|---|---|---|---|---|---|---|---|---|---|---|
| | Comparative | | | | | | | | | |
| Ingredients and pH | Example 1 | | Example 26 | | Example 27 | | Example 28 | | Example 29 | |
| 5-HT₃ receptor antagonist | granisetron | | azasetron | | tropisetron | | ramosetron | | ondansetron | |
| | hydrochloride | | hydrochloride | | hydrochloride | | hydrochloride | | hydrochloride | |
| | 147.4 mg | | 728.8 mg | | 372.2 mg | | 66 mg | | 330 mg | |
| kappa-carrageenan | 0.4 | g | 0.4 | g | 0.4 | g | 0.4 | g | — | |
| (manufactured by Sansho Co., Ltd.) | | | | | | | | | | |
| iota-carrageenan | 0.9 | g | 0.9 | g | 0.9 | g | 0.9 | g | 0.9 | g |
| (manufactured by Sansho Co., Ltd.) | | | | | | | | | | |
| low-methoxyl pectin | — | | — | | — | | — | | — | |
| locust bean gum | 0.4 | g | 0.4 | g | 0.4 | g | 0.4 | g | 0.4 | g |
| (CP Kelco Co., Ltd., San-Ei Gen | | | | | | | | | | |
| F.F.I., Inc.) | | | | | | | | | | |
| dextrin | 5 | g | 5 | g | 5 | g | 5 | g | 5 | g |
| (Japanese Pharmacopoeia 14: Nippon | | | | | | | | | | |
| Starch Chemical Co., Ltd. ) | | | | | | | | | | |
| citric acid | 0.3 | g | 0.8 | g | 0.12 | g | 0.5 | g | 1 | g |
| (Japanese Pharmacopoeia 14) | | | | | | | | | | |
| sodium citrate | 1 | g | 1 | g | 1 | g | 0.8 | g | 0.5 | g |
| (Japanese Pharmacopoeia 14) | | | | | | | | | | |
| sodium polyacrylate | 0.132 | g | 0.132 | g | 0.132 | g | 0.132 | g | 0.132 | g |
| (manufactured by Nihon Junyaku | | | | | | | | | | |
| Co., Ltd.) | | | | | | | | | | |
| D-sorbitol | 56 | g | 56 | g | 56 | g | 56 | g | 56 | g |
| (Japanese Pharmacopoeia 14) | | | | | | | | | | |
| glycerin | 27 | g | 27 | g | 27 | g | 27 | g | 27 | g |
| (Japanese Pharmacopoeia 14) | | | | | | | | | | |
| sodium pyrosulfite | 0.1 | g | 0.1 | g | 0.1 | g | 0.1 | g | 0.1 | g |
| (manufactured by Daito Chemical | | | | | | | | | | |
| Co., Ltd.) | | | | | | | | | | |
| propyl parahydroxybenzoate | 0.5 | g | 0.5 | g | 0.5 | g | 0.5 | g | 0.5 | g |
| (Japanese Pharmacopoeia 14) | | | | | | | | | | |

| | | | | | |
|---|---|---|---|---|---|
| flavor | trace amount | trace amount | trace amount | trace amount | trace amount |
| pH | 8 | 7 | 6 | 5 | 3 |

Purified water was added to prepare 198 g in total and then filled in a container.

TABLE 7

| | Blending quantity | | | | | | | | | |
|---|---|---|---|---|---|---|---|---|---|---|
| Ingredients and pH | Example 30 | | Example 31 | | Example 32 | | Example 33 | | Example 34 | |
| 5-HT₃ receptor antagonist | granisetron | | azasetron | | tropisetron | | ramosetron | | ondansetron | |
| | hydrochloride | | hydrochloride | | hydrochloride | | hydrochloride | | hydrochloride | |
| | 147.4 mg | | 728.8 mg | | 372.2 mg | | 66 mg | | 330 mg | |
| kappa-carrageenan | 0.4 | g | 0.4 | g | 0.4 | g | 0.4 | g | 0.4 | g |
| (manufactured by Sansho Co., | | | | | | | | | | |
| Ltd.) | | | | | | | | | | |
| iota-carrageenan | 0.9 | g | 0.9 | g | 0.9 | g | 0.9 | g | 0.9 | g |
| (manufactured by Sansho Co., | | | | | | | | | | |
| Ltd.) | | | | | | | | | | |
| locust bean gum | 0.4 | g | 0.4 | g | 0.4 | g | 0.4 | g | 0.4 | g |
| (CP Kelco Co., Ltd., San-Ei Gen | | | | | | | | | | |
| F.F.I., Inc.) | | | | | | | | | | |
| dextrin | 5 | g | 5 | g | 5 | g | 5 | g | 5 | g |
| (Japanese Pharmacopoeia 14: Nippon | | | | | | | | | | |
| Starch Chemical Co., Ltd.) | | | | | | | | | | |
| citric acid | 0.12 | g | 0.12 | g | 0.12 | g | 0.12 | g | 0.12 | g |
| (Japanese Pharmacopoeia 14) | | | | | | | | | | |
| sodium citrate | 1 | g | 1 | g | 1 | g | 1 | g | 1 | g |
| (Japanese Pharmacopoeia 14) | | | | | | | | | | |
| sodium polyacrylate | 4 | mg | 4 | mg | 4 | mg | 4 | mg | 4 | mg |
| (manufactured by Nihon | | | | | | | | | | |
| Junyaku Co., Ltd.) | | | | | | | | | | |
| D-sorbitol | 56 | g | 56 | g | 56 | g | 56 | g | 56 | g |
| (Japanese Pharmacopoeia 14) | | | | | | | | | | |
| glycerin | 27 | g | 27 | g | 27 | g | 27 | g | 27 | g |
| (Japanese Pharmacopoeia 14) | | | | | | | | | | |
| propyl parahydroxybenzoate | 0.5 | g | 0.5 | g | 0.5 | g | 0.5 | g | 0.5 | g |
| (Japanese Pharmacopoeia 14) | | | | | | | | | | |

| | | | | | |
|---|---|---|---|---|---|
| flavor | trace amount | trace amount | trace amount | trace amount | trace amount |
| pH | 6.5 | 6.5 | 6.5 | 6.5 | 6.5 |

Purified water was added to prepare 198 g in total and then filled in a container.

### Test Example 1

Effects of pH on the stability of the jellied composition of the present invention

Stability tests were conducted on pharmaceutical preparations of the jellied compositions obtained in Examples 26 to 29 and Comparative Example 1 (adjusted to pH 3 to 8 by controlling the types and quantities of the gelatinizing agents and buffers) encapsulated in ampules and those filled in sticks, respectively.

(1) For products encapsulated in ampules, preservation tests were carried out at 70° C. for 2 weeks.

(2) For products filled in sticks, preservation tests were carried out at 40° C. 75% RH for one month or two months.

The compositions after the preservation were observed with respect to their colors. The observation results are listed in Table 8.

TABLE 8

| | Immediately | Stick product | Ampule product | Stick product | Stick product |
|---|---|---|---|---|---|
| | after the | at room temp. | at 70° C. | at 40° C. 75% | at 40° C. 75% |
| | production | for 2 months | for 2 weeks | for 1 month | for 2 months |
| Example 26(pH 7) | colorless | colorless | Colorless | Colorless | Colorless |
| Example 27(pH 6) | Colorless | colorless | Colorless | Colorless | Colorless |
| Example 28(pH 5) | Colorless | Colorless | Colorless | Colorless | Colorless |
| Example 29(pH 3) | Colorless | Colorless | Colorless | Colorless | Colorless |
| Comparative | Colorless | Colorless | faint yellow | Colorless | faint yellow |
| Example 1(pH 8) | | | | | |

As shown in Table 8, each of the pharmaceutical compositions for oral administration of Examples 26 to 29 and Comparative Example 1, both the stick product stored at room temperature and the stick product stored at 40° C. 75% RH for one month was remained in colorless. However, for an ample product stored at 70° C. for two weeks and the stick products stored at 40° C. 75% RH, for 2 months, those of Examples 26 to 29 remained in colorless while those of Comparative Examples 1 were colored. That is, it is evident that the compositions at pH 3 to 7 are more stable than the composition at pH 8.

### Test Example 2

Effects of a reductant on the stability of the jellied composition of the present invention

Pharmaceutical preparations for oral administration of the jellied compositions obtained in Examples 1 to 5 and the jellied composition obtained in Examples 30 to 34 (similar to those of Examples 1 to 5 except the absence of a reductant: sodium pyrosulfite) encapsulated in ampules (products encapsulated in ampules) were stored at 80° C. Those obtained immediately after the preparing, and those stored for 5 hours and 10 hours at 80° C. were observed with respect to their color tones, respectively. The results are listed in Table 9.

TABLE 9

| | Immediately | | |
|---|---|---|---|
| | after the | After 5 | After 10 |
| | preparing | hours | hours |
| Example 1 | Colorless | Colorless | Colorless |
| Example 2 | Colorless | Colorless | Colorless |
| Example 3 | Colorless | Colorless | Colorless |
| Example 4 | Colorless | Colorless | Colorless |
| Example 5 | Colorless | Colorless | Colorless |
| Example 30 | Colorless | slightly yellow | faint yellow |
| Example 31 | Colorless | faint yellow | faint yellow |
| Example 32 | Colorless | faint yellow | yellow |
| Example 33 | Colorless | Colorless | slightly yellow |
| Example 34 | Colorless | faint yellow | faint yellow |

As shown in Table 9, the colors of the respective compositions of Examples 30 to 34 were changed with times, while the compositions of Examples 1 to 5 remained in colorless without any change. This indicates that the addition of a reductant causes the composition to be stabilized.

### Test Example 3

Observations of the jellied compositions of the present invention

The pharmaceutical preparations for oral administration prepared by encapsulating the compositions obtained in Examples 1 to 25 in ampules and those prepared by filling them in sticks were subjected to a stability test, respectively. The test conditions were the same manner as those in Test Example 1. Each of the compositions of the pharmaceutical preparations for oral administration shows no change in color and remains in colorless. Besides, no external appearance such as synthesis was observed.

### INDUSTRIAL APPLICABILITY

The present invention can provide a pharmaceutical preparation in a dosage form that can prevent cancer patients, elderly, or dysphagia patients, who require oral administration of 5-HT.₃ receptor antagonists, from reflex vomiting. In addition, the present invention can provide a medicine for oral administration which is easy to conduct self-medication by encapsulating or packing the pharmaceutical preparation in a container which is easy to use in administration.

1. A jellied pharmaceutical composition for oral administration, comprising a 5-HT.₃ receptor antagonist, a gelatinizing agent, and water, wherein the composition has a pH of 7 or less.

2. The pharmaceutical composition for oral administration according to claim 1, further comprising a reductant.

3. The pharmaceutical composition for oral administration according to claim 1, wherein the 5-HT₃ receptor antagonist is azasetron, granisetron, tropisetron, ramosetron or ondansetron, or an organic acid salt or inorganic acid salt thereof.

4. The pharmaceutical composition for oral administration according to claim 1, wherein the gelatinizing agent is carrageenan, pectin, agar, alginic acid, sodium alginate, gelatin, manna, Kodak, konjakmannan, glucomannan, chitosan, xanthan gum, tamarind seed polysaccharide, gellan gum, karaya gum or cassia gum, or a combination of two or more thereof.

5. The pharmaceutical composition for oral administration according to claim 1, wherein
the gelatinizing agent comprises carrageenan and the carrageenan is kappa (κ)-carrageenan and/or iota (ι)-carrageenan.

6. The pharmaceutical composition for oral administration according to claim 1, further comprising a thickener, wherein the thickener is locust bean gum, gum arabic, tragacanth, dextrin, dextran, arabinogalactan, pullulan, carmellose sodium, hydropropyl cellulose, hydroxyethyl methyl cellulose, methyl cellulose, carboxmethyl cellulose, copolydone, polyvinylpyrrolidone, carboxyvinyl polymer, sodium polyacrylate or macrogol, or a combination of two or more thereof.

7. The pharmaceutical composition for oral administration according to claim 1, further comprising a water-soluble salt of potassium or calcium.

8. A medicine for oral administration, comprising the pharmaceutical composition for oral administration according to claim 1 and a light-blocking type container containing the pharmaceutical composition.
